(19) United States
(12) Patent Application Publication
(10) Pub. No.: US 2023/0288409 A1
(43) Pub. Date: Sep. 14, 2023

(54) Reusable Cartridges for Detecting Analytes in Solution

(71) Applicant: Genentech Inc., South San Francisco, CA (US)

(72) Inventor: Samuel Benjamin Pollock, South San Francisco, CA (US)

(73) Assignee: Genentech Inc., South San Francisco, CA (US)

(21) Appl. No.: 18/319,618

(22) Filed: May 18, 2023

Related U.S. Application Data
(63) Continuation of application No. PCT/US21/59819, filed on Nov. 18, 2021.
(60) Provisional application No. 63/116,575, filed on Nov. 20, 2020.

(51) Int. Cl. G01N 33/543

(52) U.S. Cl. CPC G01N 33/54366; G01N 2333/70539

(57) ABSTRACT

This disclosure relates to methods of preparing re-usable and dry-able cartridges comprising matrices comprising a detection molecule such as an antibody, for detection or enrichment of an analyte in a sample, such as an antigen recognized by an antibody. In some embodiments, the cartridge comprises a tube or well that holds the matrix. In some embodiments, the matrix or associated cartridges may be stored wet or dry. The disclosure also relates to the cartridges and methods of their use.

### CROSS-REFERENCE TO RELATED APPLICATIONS

This application claims the benefit of priority to U.S. Provisional Application No. 63/116,575, filed Nov. 20, 2020, which is incorporated by reference herein in its entirety for any purpose

### FIELD

This disclosure relates to methods of preparing re-usable and dry-able cartridges comprising matrices comprising a detection molecule such as an antibody, for detection or enrichment of an analyte in a sample, such as an antigen recognized by an antibody. In some embodiments, the cartridge comprises a tube or well that holds the matrix. In some embodiments, the matrix or associated cartridges may be stored wet or dry. The disclosure also relates to the cartridges and methods of their use.

### BACKGROUND

Various commercially available matrices may be used for detecting particular analytes in solution samples. For example, matrices comprising materials coated with protein A, protein G, and/or protein L can be used to trap antibody constant regions on a matrix, thus detecting and enriching them compared to other components in a solution. Matrices can be coated with antibodies, either directly or through protein A, protein G, and/or protein L on the matrix, for example, to detect or enrich antigen analytes in a sample. Matrices can be coated with antigens, for example, to detect or enrich antibodies to those antigens. Matrices coated with haptens may also be used to detect analytes in a solution. Materials forming matrices are varied, such as resins, beads, etc.

However, many matrices and cartridges holding such matrices, once they have been used to detect an analyte in a solution, particularly in a cell lysate, for example, cannot be re-used efficiently and are discarded after a single use. As a result, use of such cartridges, for example in regular screening experiments, can be extremely costly as protein reagents for coating the cartridges may need to be continuously available in large quantities, as well as considerably time consuming, as fresh cartridges may need to be prepared on a regular basis. For example, particular detection molecules may need to be prepared and then attached to the matrix before it can be used, which may require a number of process steps. And some of the necessary reagents, such as commercial antibodies or antigens, may be very costly to purchase in the quantities needed to prepare sufficient coated matrix. Thus, if the matrix can be re-used, much time and resources might be saved.

The disclosure herein describes, inter alia, methods for preparing cartridges comprising a matrix that are re-usable, for example, several times, and also that can be stored in a dry state between uses for easier handling, for the detection of analytes in solutions, including in complex solutions such as cell lysates, as well as the associated re-usable cartridges. The disclosure herein also describes exemplary methods of using cartridges described herein for detection of particular analytes in a solution sample, such as major histocompatibility complex Class I (MHC-I) molecules and other proteins in biological fluids and cell lysates.

### SUMMARY

This disclosure includes, inter alia, a reusable cartridge for detecting an analyte in a solution, wherein the cartridge comprises: a) a matrix, b) detection molecules attached to the matrix, wherein the detection molecules specifically bind to the analyte, and wherein the detection molecules are optionally cross-linked to the matrix; and c) at least one cryoprotectant, wherein the cartridge can be used at least 10, 20, 50, or 100 times for detection of the analyte in a solution, and wherein, after each use the matrix is optionally stored with cryoprotectant in a dry state. In some aspects, the cartridge is a tube open at both ends, a tube open at one end, a well, a plate with or without wells, or a chip capable of containing the matrix. In some aspects, the matrix comprises particles (e.g. beads, grains, chips, or pellets) comprising silica or agarose, wherein the particles are optionally magnetic. In some cases, the matrix comprises protein A, protein G, and/or protein L. In some cases, the detection molecules are antibodies. In some cases, the detection molecules are cross-linked to the matrix. In some cases, more than one type of detection molecule is attached to the matrix, e.g., two different antibodies for detecting two different antigen analytes in a solution. In some aspects, the detection molecules are cross-linked to the matrix with a crosslinker selected from selected from dimethylpimelimidate (DMP), a cyanate ester, an NHS ester, azlactone, carbonyl diimidazole (CDI), maleimide, iodoacetyl, pyridyl disulfide, hydrazide, or a carbodiimide. In some aspects, the analyte detectable by the detection molecules on the matrix is a protein or peptide. For example, in some cases, the analyte is a major histocompatibility complex I (MHC-I) molecule. In some cases, the cartridge can be used at least 10 times for detection of a protein analyte in a cell lysate or biological fluid. In some cases, the reusable cartridge is stored in the dry state. In some aspects, the cartridge can be stored in the dry state after preparation, and then used at least 10 times for detection of a protein analyte in a cell lysate or biological fluid after storage in the wet state after each use. In some aspects, the cartridge can be used at least 10 times for detection of a protein analyte in a cell lysate or biological fluid after storage in the dry state after each use. In some aspects, the cartridge can be stored in the dry state after preparation, and then used at least 20 times for detection of a protein analyte in a cell lysate or biological fluid after storage in the wet state after each use. In some cases, the cartridge can be used at least 20 times for detection of a protein analyte in a cell lysate or biological fluid after storage in the dry state after each use. In some aspects, the cartridge can be stored in the dry state after preparation, and then used at least 50 times for detection of a protein analyte in a cell lysate or biological fluid after storage in the wet state after each use. In some cases, the cartridge can be used at least 50 times for detection of a protein analyte in a cell lysate or biological fluid after storage in the dry state after each use. In some aspects, the cartridge can be stored in the dry state after preparation, and then used at least 100 times for detection of a protein analyte in a cell lysate or biological fluid after storage in the wet state after each use. In some cases, the cartridge can be used at least 100 times for detection of a protein analyte in a cell lysate or biological fluid after storage in the dry state after each use. In some aspects, the matrix is stored at acidic pH. In some aspects, the matrix is stored at 2-8° C. In some aspects, the matrix is stored in a wet state. In some aspects, the matrix is stored at acidic pH, and optionally at 2-8° C. In some cases, the cryoprotectant comprises one or more of sucrose, trehalose, ethylene glycol, propylene glycol, glycerol, 2-methyl-2,4-pentanediol (MPD), or dimethyl sulfoxide (DMSO).

The present disclosure also relates to methods of preparing a previously used cartridge, for instance, as described above or otherwise herein, for re-use, comprising, after analyte has been eluted from the cartridge, washing the matrix in 1-10% acetic acid, 1% formic acid, or 1% trifluoroacetic acid (TFA), washing the matrix in a buffer comprising cryoprotectant, and allowing the matrix to dry. In some aspects, the cryoprotectant comprises one or more of sucrose, trehalose, ethylene glycol, propylene glycol, glycerol, 2-methyl-2,4-pentanediol (MPD), or dimethyl sulfoxide (DMSO). In some cases, the matrix is allowed to dry by heating the cartridge to at least 30° C., followed by storage at room temperature for at least 1 hour. In some cases, the matrix is allowed to dry by heating the cartridge to 37° C., followed by storage at room temperature for at least 1 hour. In some cases, the matrix is allowed to dry, the cartridge is stored in a dry state at 2-8° C. until re-use.

The present disclosure further relates to methods of preparing a reusable cartridge, as described above or otherwise herein, comprising obtaining a cartridge comprising a matrix, contacting the matrix with the detection molecules, and crosslinking the detection molecules to the matrix.

The disclosure further relates to methods of detecting an analyte in a solution, comprising contacting the matrix of a reusable cartridge described above or otherwise herein with a solution comprising the analyte, and optionally eluting the analyte from the cartridge. In some aspects, the analyte is a peptide or protein. In some aspects, the analyte is an MHC-I molecule. In some cases, the solution is a biological fluid or cell lysate. In some cases, the solution is filtered or treated to remove cell debris and membranous materials prior to contact with the matrix. In some cases, the cartridge has previously been used to detect the analyte at least 1, 2, 3, 4, 5, 6, 7, 8, 9, 10, 20, 50, or 100 times prior to its use in the method. In some cases, following elution of analyte, the cartridge is treated by methods of preparing a previously used cartridge herein, such as those described above.

The present disclosure also encompasses kits comprising at least one reusable cartridge as described herein, such as those described above. In some aspects, a kit comprises at least one cartridge as described herein as well as: (a) at least one buffer; (b) at least one control cartridge without detection molecule or with a control detection molecule; (c) reagents for preparing the cartridge for storage and reuse; and/or (d) instructions for use

It is to be understood that both the foregoing general description and the following detailed description are exemplary and explanatory only and are not restrictive of the claims. The accompanying drawings, which are incorporated in and constitute a part of this specification, illustrate certain embodiments or aspects, and together with the description, serve to explain the principles described herein.

### BRIEF DESCRIPTION OF THE DRAWINGS

The present US provisional application contains at least one drawing executed in color. If the present US provisional application is made available to the public in future due to the publication of a nonprovisional or international application claiming priority to it, then copies of this provisional application with color drawings will be provided by the Office upon request and payment of the necessary fee.

FIGS. 1A-E show an overview of the use of reusable cartridges as described herein in a method for detecting MHC-I complexes. FIG. 1A provides an overview of the stages in MHC-I associated peptide (MAP) endogenous processing and presentation enrichment workflow, and mass spectrometry-based analysis and quantification. In canonical presentation, intracellular proteins are polyubiquitinated within the cell and translocated to the proteasome for degradation. The resulting peptide fragments are transported into the endoplasmic reticulum via TAP proteins. These are then loaded into MHC-I complexes (consisting of an MHC-I heavy chain and B2M), further trimmed by ERAP, and the stable MHC-I peptide complex is translocated to the cell surface. The display level of each unique peptide is determined by its abundance, degradation rate, and affinity for the MHC-I allele(s) in a given cell. Enrichment involves applying lysate containing MHC-I complexes to a solid support containing anti-MHC-I antibodies, washing away unbound contaminants, and then eluting the MHC-I proteins and associated peptides via acid treatment. Analysis involves peptide isolation and desalting, chromatographic separation, and analysis by mass spectrometry followed by data processing involving identification and quantification steps. FIG. 1B depicts small and large capacity cartridges and provides a table of small and large cartridge characteristics, including recombinant protein capacities, cellular abundance, and amount to be loaded on customized antibody cartridges. FIG. 1C depicts unique MHC-I peptides identified using a standard, single-use AssayMAP™ enrichment workflow, separated by species and effective cell count. FIG. 1D provides an antibody cartridge reuse scheme and circular enrichment workflow. Protein A cartridges are loaded with antibody and cross-linked, used to enrich MHC-I complexes, and washed before acid elution. Cartridges are then cleaned via priming with acid and TBS, stored at 4° C., and re-used in a similar manner. FIG. 1E shows unique peptide count observed after consecutive uses of custom antibody cartridges, using GRANTA lysate, on always-wet vs. dried then re-wetted cartridges.

FIG. 2 shows gel electrophoresis comparing analytes enriched using re-used always wet vs. dried and re-wetted cartridges. Lanes 1-3, at left, show a titration of a purified HLA protein standard. Numbers on the Y-axis provide positions of molecular weight ladder bands, in kiloDaltons. Lanes 4-6 show the level of HLA standard eluted from wet-stored cartridges, while the right-hand three lanes 7-9 show the level of HLA eluted from dry-stored/re-wetted cartridges. The similar levels in each of lanes 4-9 show that dry-stored and re-wetted cartridges are able to detect or enrich the standard protein similarly to the cartridges that were stored always wet.

FIG. 3 shows Coomassie electrophoresis gel data used to create a calibration curve and estimated capacity for recombinant MHC-I complexes on a previously used large capacity antibody cross-linked cartridge.

FIGS. 4A-B show the effect of cross-linking on enrichment of MHC-I molecules from cartridges. FIG. 4A depicts the time at which recombinant HLA and B2M proteins (the subunits of MHC-I molecules) and a sample peptide molecule and an antibody recognizing MHC-I molecules each should elute in a liquid chromatography mass spectrometry (LC-MS) procedure following enrichment of MHC-I molecules on cartridges. FIG. 4B shows a comparison of LC-MS runs following MHC-I enrichment on cartridges to which the MHC-I-recognizing antibody is either cross-linked (top) or not cross-linked (bottom). The results show that cross-linking has no apparent effect on MHC-I enrichment (compare largest peak in “cross-linked” to size of peak directly below in “non-linked.”) The antibody peak (largest peak in “non-linked”) is not visible in the cross-linked elution, demonstrating antibody retention. A second elution from the column (elution 2) shows that cross-linking also does not lead to additional non-specific binding to the cartridge material.

FIG. 5 shows a bar graph depicting an analysis of the viscosity of lysate based on volume retained on top of a spin filter under various conditions described in the working examples below and below the bars of the graph. Under the conditions described (500 μL, placed on a 0.45 um Costar filter and spun at 16,000 g for 1 min at 4° C.), water and lysis buffers show no volume retained (negative control), while room temperature day-old MC38 lysate (50M cells per mL of B-buffer, positive control), shows retention of over 400 μL. Fresh GRANTA lysate (50M cells per mL of B-buffer) shows no retention, while 4 hours at room temperature (typical, un-optimized loading condition), shows a major increase in viscosity. Lowering the temperature to 4° C. reduces aggregation in a manner that is maintained over 18 hrs. If lysate is flash frozen with PBS, sucrose (1M starting, 200 mM final), or glycerol (50% glycerol starting, 10% final) and then thawed, viscosity is low, even after 4 hr at room temperature (however 24 hr at room temperature leads to a large increase in viscosity). Combining sucrose and glycerol preservation (leading to an even more dilute lysate solution) allows for a freeze thaw with no apparent retention immediately, or after 4 hrs at room temperature.

FIGS. 6A-H show several Venn diagrams depicting the overlap in composition between different cartridge enrichments of the same batch of GRANTA lysate (see Example 2). Peptides in each data set were weighted for abundance using TIC signal (so that far more abundant peptides contributed more to the calculated overlap) but were not corrected for abundance (so that different amounts of signal between LC-MS/MS runs was preserved in the calculation). Numbers represent the number of unique peptides detected in a MS/MS run from a solution comprising MHC-I complexes. Comparisons made here include between “wet” and “dry” cartridges, between replicate cartridges of the same type, and over re-uses. Specifically, FIG. 6A compares a first use (first elution or E1) of a cartridge that was previously stored wet (E1W1) to one that was stored dry (E1D1). FIG. 6B and FIG. 6C show similar comparisons using different sets of wet-stored and dry-stored cartridges (W2/D2 and W3/D3). FIG. 6D shows overlap of results from a first elution (i.e. first use; E1) of three different cartridges of the same type, all dry-stored (D1, D2, D3). FIG. 6E shows overlap from a second elution (E2) for three different wet-stored cartridges (W1, W2, or W3). FIGS. 6F, 6G, and 6H show comparisons of first, fifth, and ninth uses (E1, E5, and E9) on three different dry-stored cartridges (D1 (FIG. 6F), D2 (FIG. 6G), and D3 (FIG. 6H)).

### FURTHER DESCRIPTION OF CERTAIN EMBODIMENTS

### Definitions

Unless otherwise defined, scientific and technical terms used in connection with the present invention shall have the meanings that are commonly understood by those of ordinary skill in the art.

In this application, the use of “or” means “and/or” unless stated otherwise. In the context of a multiple dependent claim, the use of “or” refers back to more than one preceding independent or dependent claim in the alternative only. Also, terms such as “element” or “component” encompass both elements and components comprising one unit and elements and components that comprise more than one subunit unless specifically stated otherwise.

As described herein, any concentration range, percentage range, ratio range or integer range is to be understood to include the value of any integer within the recited range and, when appropriate, fractions thereof (such as one tenth and one hundredth of an integer), unless otherwise indicated.

Units, prefixes, and symbols are denoted in their Système International de Unites (SI) accepted form. Numeric ranges are inclusive of the numbers defining the range. The headings provided herein are not limitations of the various aspects of the disclosure, which can be had by reference to the specification as a whole. Accordingly, the terms defined immediately below are more fully defined by reference to the specification in its entirety.

As utilized in accordance with the present disclosure, the following terms, unless otherwise indicated, shall be understood to have the following meanings:

A “sample” as used herein refers to any specimen that may contain an analyte for detection or enrichment. A “solution” herein refers to a sample in liquid form.

An “analyte” herein is used in the broadest sense to refer to a molecule or substance that may be found in a sample or solution and that is intended to be bound to (i.e., recognized by or detected by) the detection molecules on a matrix, and thereby detected or enriched. An analyte in some cases can be a protein or peptide or another type of biological molecule.

A “detection molecule” as used herein is any molecule that specifically binds, either directly or indirectly, to an analyte. A detection molecule may be, for example, an antibody, an antigen molecule, a hapten molecule, or any molecule that specifically binds the analyte, and that is compatible with the matrix. As used herein, the “detection molecules” attached to a matrix may be molecules of a single molecular species, or alternatively, can be a mixture of molecules of two or more different molecular species.

In some embodiments, the analyte and/or detection molecule may be a protein or polypeptide or peptide molecule. The terms “polypeptide” and “protein” are used interchangeably and refer to a polymer of amino acid residues. Such polymers of amino acid residues may contain natural and/or non-natural amino acid residues, and include, but are not limited to, peptides, oligopeptides, dimers, trimers, and multimers of amino acid residues. The terms also include polymers of amino acids that have modifications such as, for example, glycosylation, sialylation, and the like, or that are complexed with other molecules. A “peptide” herein is a relatively short polymer of amino acids, such as on the order of 4 to 50 amino acids.

A protein may, in some cases, be an antibody. The term “antibody” or “Ab” herein is used in the broadest sense and encompasses various antibody structures, including but not limited to monoclonal antibodies (“mAb”), polyclonal antibodies, multispecific antibodies (e.g., bispecific antibodies), antibody conjugates, and antigen binding fragments, so long as they exhibit the desired antigen-binding activity. As used herein, the term refers to a molecule comprising at least complementarity-determining region (CDR) 1, CDR2, and CDR3 of a heavy chain and at least CDR1, CDR2, and CDR3 of a light chain, wherein the molecule is capable of binding to an antigen. The term also encompasses antigen binding fragments. The term “antigen binding fragment” includes, but is not limited to, fragments of antibodies that are capable of binding antigen, such as Fv, single-chain Fv (scFv), Fab, Fab′, and (Fab′)₂. In contrast, a “full length antibody” refers to an antibody molecule comprising all of its normal variable and constant region portions. An antibody conjugate may comprise a full length antibody or antigen binding fragment that is attached directly or indirectly to another molecule. Like other proteins and peptides, in some embodiments, antibodies may contain various types of post-translational modifications, such as glycosylations.

“Detection” herein is used in the broadest sense to encompass selective binding of an analyte in a solution over other molecules in the solution, for example, to determine whether the analyte is present in the solution, as well as to isolate an analyte from other components of the solution, thereby enriching the analyte. In some cases, methods herein are used to detect the presence of an analyte. In some cases, methods herein are used to “enrich” or “isolate” an analyte in comparison to other molecules in a solution, for example, so that further analyses may be performed on the analyte. The terms “enrich” and “isolate” are used interchangeably in this context herein.

A “cartridge” is used in a broad sense to refer to an overall product that contains or holds a “matrix.” A “cartridge” may have any useful shape or structure and may be made from any useful material, such as glass or plastic material or polymeric material. A “cartridge” may comprise any useful material that is configured or shaped so that it can enclose or contain the matrix and hold it in place. In general, the cartridge allows for fluids to flow across and/or through the matrix. In some embodiments, the matrix may be positioned in a tube-shaped or cylindrical cartridge with openings at both ends (e.g., as in a pipette tip, capillary, column, or other configuration), to facilitate transfer of liquid solutions to and from the cartridge during use, such as via an automated liquid dispensing device. In some cases, a cartridge may have a U-shape, or comprise a well, allowing transfer of fluids from one end of the matrix, or it may have a plate-like, or a flat, chip-like structure in or on which the matrix is located, allowing fluids to flow across its surface, for example.

A “matrix” broadly refers to a substance to which detection molecules may be attached. Suitable matrices herein include chromatography matrices such as C4, C8, or C18, or various types of beads, particles, or resins, such as silica, polymeric beads and/or metallic beads. In some cases, the matrix may be coated with a protein, such as an antibody, antigen, antibody constant region binding protein, or hapten that may recognize a detection molecule. For example, protein A, G, or L can be present on the matrix to recognize an antibody detection molecule, or streptavidin could be used on the matrix to recognize a biotinylated detection molecule. In a matrix composed of beads or resin particles, the particles may be of any appropriate shape or size so long as they can hold the detection molecules, such as spherical or roughly spherical beads, or other shapes such as pellets, chips, tablets, etc. In some embodiments, the matrix particles may be magnetic.

Detection molecules may be “attached” to the matrix covalently or noncovalently. In some embodiments, a crosslinking reagent may be used in order to crosslink the detection molecules to the matrix. Attachment may also be direct or indirect, i.e. either without or with an intervening molecule between the matrix materials and the detection molecule.

A cartridge may be “reusable” if it can be used to detect analyte in a first solution, then washed and re-equilibrated to detect analyte in a second solution without significant, observable difference in the ability to detect an analyte in multiple tests using the same solution.

A matrix herein may be stored in a wet or dry state. Storage in a “wet state” means that the matrix is maintained submerged within a liquid solution, such as a buffer solution. Storage in a “dry state” means that that the matrix is at least partially exposed to air during storage or otherwise that liquid is allowed to evaporate from the matrix before or during storage.

“Specific binding” or “specifically binds to” or “specifically recognizes” or similar terms as used herein in reference to binding between a detection molecule and an analyte mean that the binding between the molecules is at higher affinity than it would be if the binding between the analyte and detection molecule was due to nonspecific binding.

“Mass spectrometry” or “MS” refers to a technique that measures the mass to charge ratio (m/z) of one or more molecules in a sample. As used herein, “tandem MS” or “MS/MS” refers to the process by which a single ion, multiple ions, or the entire mass envelope (the precursor(s)) are moved to a fragmentation chamber and the fragmented products are then sent to a mass analyzer. Depending on the design of the mass spectrometer, the fragmentation event can happen before a single mass analyzer, between two or multiple different analyzers, or within a single mass analyzer.

### Cartridges and Methods of Preparing Cartridges for Initial Use and for Re-Use

The present disclosure relates, inter alia, to reusable cartridges for detecting an analyte in a solution, wherein the cartridges comprise a matrix, which is optionally held in place by the cartridge or by a structure comprised within the cartridge, wherein detection molecules that specifically recognize the analyte are attached to the matrix, for example by non-covalent binding to a substance in the matrix. In some cases, the detection molecules can be cross-linked to the matrix, for instance, to avoid being shed from the matrix during various treatments.

A reusable cartridge herein may be in any suitable shape for holding the matrix so that detection of analyte can take place. In some embodiments, the starting cartridge may comprise a tube or cylindrical component that acts to hold the matrix. For example, a cartridge may be in the shape of a spin or gravity flow column or pipette tip or chromatography column, allowing fluid to flow across a matrix embedded inside, either with centrifugation or by gravity flow. Alternatively, the cartridge may comprise flexible tubing or a capillary holding the matrix. In some cases, a matrix may be surrounded by a filter coarse enough to allow solution components to freely pass through but fine enough to hold the matrix materials in place. In some embodiments, the cartridge may have a tube-like or well-like structure that is open at one end, or a plate-like structure or a flat, chip-like structure that holds the matrix in place. For example, a tube, well or plate may be open on one end or on one side to allow contact with buffers and with solutions containing analyte and removal of those solutions.

In some embodiments, a single cartridge comprises more than one space, cavity, location or well comprising matrix, such that one cartridge can be used to run several different detection reactions. In other embodiments, a single cartridge comprises a single matrix component so that, to run different detection reactions in parallel, multiple cartridges are needed. In some embodiments, cartridges can be arranged into a multi-sample system, such as a 96-sample or 384-sample system or the like, for example, for automated handling and liquid dispensations.

In some embodiments, a cartridge has a volume capacity of, for example, 2 μL to 1 mL, such as 10 μL to 500 μL, 2 μL to 50 μL, 2 μL to 25 μL, 2 μL to 10 μL, 10 μL to 50 μL, 20 μL to 30 μL, 20 μL to 100 μL, 20 μL to 200 μL, or 50 μL to 500 μL, or 100 μL to 1 mL, or 200 μL to 1 mL. In some embodiments, a cartridge has a volume capacity of, for example, 5 μL or 10 μL or 20 μL or 25 μL or 50 μL or 100 μL.

In some embodiments, the matrix comprises particles of any shape, for example, such as grains or beads or chips or spheres or pellets or tablets or the like, to which the detection molecules may be attached. The particles, in some embodiments, may be made of substances such as silica or agarose or the like. In some embodiments, the particles may be magnetic, for example, to allow them to be moved during an assay if needed, or to be held in place in the cartridge. For instance, in some cases, magnetic particles could be moved in and out of a cartridge during an assay. In other embodiments the matrix is a film or sheet to which detection molecules may be attached. In some embodiments, a matrix can be held in place in a cartridge for example via a filter (e.g. in a tube with an opening at each end), or by gravity (e.g. in a tube with an opening at one end), or by hydrophobic or electrostatic interactions with the cartridge surface (e.g. a matrix sheet or film placed on a plate or chip).

In some embodiments, the detection molecules are attached directly to the matrix, for example by noncovalent binding. In some embodiments, they are attached via covalent binding to molecules in the matrix. In some embodiments, the detection molecules are attached to the matrix indirectly via an intermediate molecule that is itself attached to the matrix. For instance, in some embodiments, the matrix comprises a molecule that binds to a particular type of detection molecule, e.g. streptavidin, which binds to biotinylated molecules, or a molecule that recognizes immunoglobulins such as protein A, protein G, or protein L, or combinations of two or more of these molecules. For instance, contacting a matrix comprising protein A with an antibody detection molecule allows the incorporation of the antibody detection molecule onto the matrix through its binding by protein A on the matrix. In some embodiments, while the matrix may comprise a reagent such as streptavidin or protein A and/or G, these molecules are not the “detection molecules” as that term is used herein because they are not used to specifically recognize a particular analyte, but are merely molecules to which detection molecules herein are attached. Thus, for example, in some embodiments, if a matrix comprises protein A, a detection molecule may comprise an antibody or other immunoglobulin or Fc containing molecule that is bound by the protein A to the matrix. The protein A is used as an intermediate link between the matrix particles and the detection molecule rather than as a detection molecule itself.

In some embodiments, a detection molecule added to a matrix may also be crosslinked to the matrix. For example, a crosslinking reagent such as dimethylpimelimidate (DMP), or a cyanate ester, NHS ester, azlactone, carbonyl diimidazole (CDI), maleimide, iodoacetyl, pyridyl disulfide, hydrazide, or carbodiimide reagent, or other reagent suitable for immobilizing an affinity reagent on a matrix may be used to attach the detection molecule to the matrix. (See, e.g., www.thermofisher.com/us/en/home (slash) life-science (slash) protein-biology (slash) protein-biology-learning-center (slash) protein-biology-resource-library (slash) pierce-protein-methods (slash) covalent-immobilization-affinity-ligands.html for further discussion of how these and additional crosslinking reagents may be used to attach molecules to an agarose matrix.) In other embodiments, depending on the expected use of the cartridges and the nature of the detection molecule, crosslinking may not be needed. In some embodiments, where DMP is used as a crosslinker, 1-10 mM DMP, 1-5 mM, 1 mM, 2 mM, 3 mM, 4 mM, 5 mM, 6 mM, 7 mM, 8 mM, 9 mM, or 10 mM DMP is used for crosslinking a detection molecule to the matrix. In some embodiments herein, the concentration of DMP used in a cross-linking reaction is about one-quarter to one-fifth of the concentration normally used for the reaction.

In some embodiments, cartridges herein also comprise at least one cryoprotectant, such as sucrose, trehalose, a glycol such as ethylene glycol or propylene glycol, glycerol, 2-methyl-2,4-pentanediol (MPD), or dimethyl sulfoxide (DMSO). For example, the cryoprotectant may be in contact with the matrix, for example, to protect detection molecules and/or other matrix components from damage or denaturation during storage, such as during storage in a dry state. In some cases, the cryoprotectant comprises glucose and/or trehalose. In some cases, the cryoprotectant comprises trehalose.

Thus, in some embodiments reusable cartridges herein comprise (a) a matrix, and (b) detection molecules attached to the matrix, wherein the detection molecules specifically bind to the analyte, and wherein the detection molecules are optionally cross-linked to the matrix. In some embodiments reusable cartridges herein comprise (a) a matrix, (b) detection molecules attached to the matrix, wherein the detection molecules specifically bind to the analyte, and wherein the detection molecules are optionally cross-linked to the matrix, and (c) at least one cryoprotectant.

Certain commercially available cartridges and matrixes for analyte detection are generally intended to be used only once and discarded, and are also intended to be maintained in a wet state prior to use. (See, e.g., AssayMap® Bravo cartridges, Agilent.) The present disclosure shows that, surprisingly, cartridges described herein may be re-used many times without significant loss of signal to detect analytes in cell culture supernatants or cell lysates, and that cartridges may be stored in a dry state. In some embodiments, the cartridge is stored in the dry state. In some such cases, the cartridge comprises cryoprotectant and is stored in the dry state. In some cases, a cartridge is stored in a dry state at 2-8° C., such as in a refrigerator or cold room, or such as at 4° C. In some cases, a cartridge stored in the dry state is re-wetted prior to use for detecting analyte. In some cases, the cartridge matrix is dried by heating to at least 30° C., such as to 30-45° C., 30-37° C., 30° C., or 37° C., for example, for at least 30 minutes, for 30-60 minutes, or for 60-120 minutes, then allowed to cool at room temperature for at least 1 hour, such as at least 2 hours, at least 4 hours, at least 8 hours, at least 12 hours, or overnight, before optionally storing at reduced temperature before re-use. In some embodiments, the cartridge is stored in a wet state, for example, wherein the matrix is submerged in a buffer solution. In some cases, a cartridge is stored in a wet state at 2-8° C., such as in a refrigerator or cold room, or such as at 4° C. In some such cases, the cartridge comprises cryoprotectant and is stored in a wet state.

In some embodiments, the cartridge is stored as above at a pH of 7.0 or below. In some embodiments, the cartridge is stored at acidic pH (i.e., at a pH below 7.0). In some embodiments, the cartridge is stored at pH 4-7, such as at pH 4-6, pH 4-6.5, pH 4.5-6.5, pH 4.8-6.8, pH 5-7, pH 4-5, pH 5-6, pH 6-6.5, or pH 6-7. In some embodiments, the cartridge is stored in a buffer comprising EDTA and/or sodium azide.

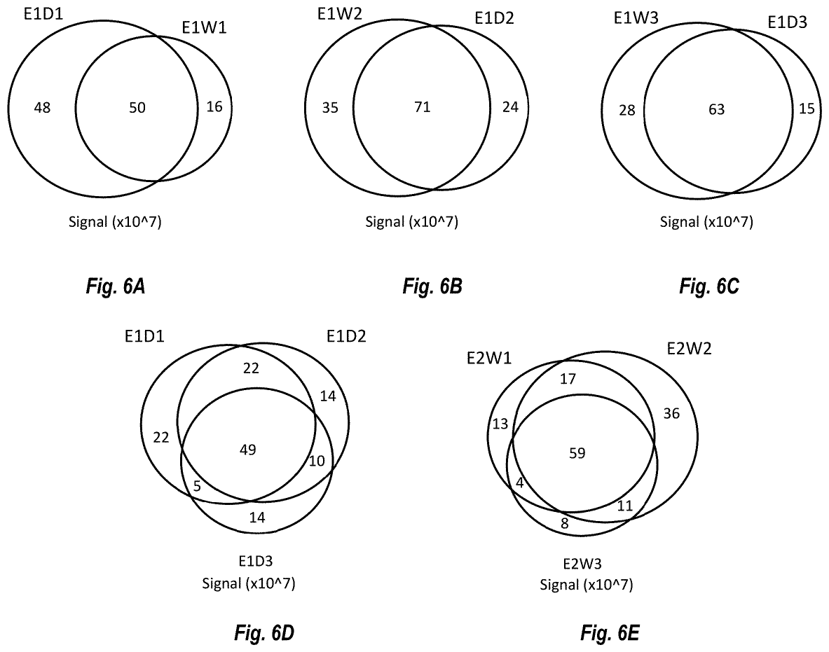

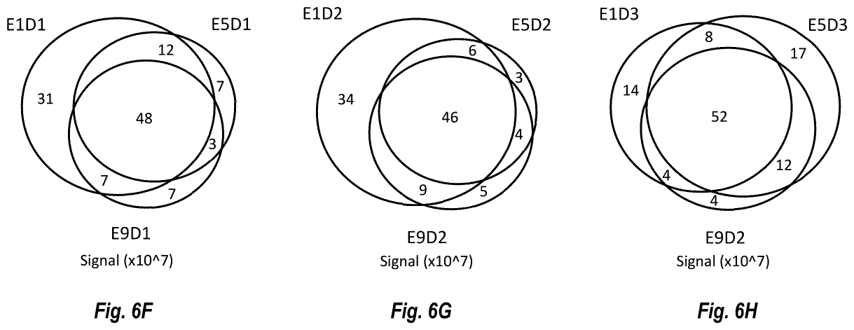

In some embodiments herein, the cartridge is a cartridge that has previously been used at least once for detection of analyte in a solution. For example, in some embodiments, a cartridge may be used to detect analyte in solution, and the detected analyte is eluted to remove it from the cartridge, and the cartridge matrix is contacted with a buffer comprising cryoprotectant for storage between uses. In some embodiments, the buffer optionally is at acidic pH and/or comprises EDTA and/or sodium azide. In some embodiments, the cartridge may be re-used at least 9 times without significant observable decrease in detection capability. For example, in some cases, the cartridge may be re-used at least 9 times with the same sample without a significant change in the composition or amount of analyte from the sample that is detected between the first and the last runs. (See, for example, FIG. 6A-H.) In some embodiments, the cartridge may be re-used at least 10, 20, 50, or 100 times without significant observable decrease in detection capability. For example, in some embodiments, a cartridge with a matrix comprising an antibody detection molecule, after at least 10, 20, 50, or 100 uses, remains able to elute at least 90% of a protein analyte from a cell lysate or biological fluid compared to the analyte eluted in a first use. In some embodiments, a cartridge with a matrix comprising an antibody detection molecule, after at least 10, 20, 50, or 100 uses, remains able to elute at least 95% of a protein analyte from a cell lysate or biological fluid compared to the analyte eluted in a first use. In some embodiments, a cartridge with a matrix comprising an antibody detection molecule, after at least 10, 20, 50, or 100 uses, remains capable of detecting an enriching a protein analyte in a biological sample with at least 90% of the efficiency as that of a cartridge in its first use. In some embodiments, a cartridge with a matrix comprising an antibody detection molecule, after at least 10, 20, 50, or 100 uses, remains capable of detecting an enriching a protein analyte in a biological sample with at least 95% of the efficiency as that of a cartridge in its first use. In some such cases, the cartridge is stored in the dry state with cryoprotectant between each use. In some such cases, the cartridge is stored in a wet state with or without cryoprotectant between each use. In some cases, a cartridge is stored either dry or wet at 2-8° C., such as in a refrigerator or cold room, or such as at 4° C. between uses. In some cases, it is stored with a buffer of acidic pH and/or comprising EDTA and/or sodium azide between each use.

In some embodiments, the cartridge is one that has been used at least twice for detection of analyte in solution. In some embodiments, the cartridge is one that has been used at least three times for detection of analyte in solution. In some embodiments, the cartridge is one that has been used at least four times for detection of analyte in solution. In some embodiments, the cartridge is one that has been used at least five times for detection of analyte in solution. In some embodiments, the cartridge is one that has been used at least six times for detection of analyte in solution. In some embodiments, the cartridge is one that has been used at least seven times for detection of analyte in solution. In some embodiments, the cartridge is one that has been used at least eight times for detection of analyte in solution. In some embodiments, the cartridge is one that has been used at least nine times for detection of analyte in solution. In some embodiments, the cartridge is one that has been used at least ten times for detection of analyte in solution. In some embodiments, the cartridge is one that has been used at least 20 times for detection of analyte in solution. In some embodiments, the cartridge is one that has been used at least 50 times for detection of analyte in solution. In some embodiments, the cartridge is one that has been used at least 100 times for detection of analyte in solution. In some such cases, the cartridge has been stored in the dry state with cryoprotectant following the prior use or uses. In some such cases, the cartridge has been stored in a wet state with or without cryoprotectant after each use. In some cases, a cartridge has been stored either dry or wet at 2-8° C., such as in a refrigerator or cold room, or such as at 4° C. between uses. In some cases, it is stored wet with a buffer of acidic pH and/or comprising EDTA and/or sodium azide between each use. In some cases, it is stored dry with cryoprotectant between each use, but is dried after having been treated with a buffer of acidic pH comprising EDTA and/or sodium azide.

In some cases where a cartridge is stored in the dry state at 2-8° C., such as in a refrigerator or cold room, or such as at 4° C., the cartridge may be stored for at least 24 hours, at least 48 hours, at least 1 week, at least 2 weeks, at least 1 month, at least 3 months, or at least 6 months. In some cases, it can be stored at 2-8° C., such as in a refrigerator or cold room, or such as at 4° C., for at least 24 hours, at least 48 hours, at least 1 week, at least 2 weeks, at least 1 month, at least 3 months, or at least 6 months in a dry state after addition of cryoprotectant. Further, in some embodiments, it can be stored under the above conditions after prior treatment with an acidic buffer comprising EDTA and/or sodium azide prior to drying out. In some cases where a cartridge is stored wet, it can be stored at 2-8° C., such as in a refrigerator or cold room, or such as at 4° C. between uses for at least 24 hours, at least 48 hours, at least 1 week, at least 2 weeks, at least 1 month, at least 3 months, at least 6 months, or at least 1 year in a buffer at acidic pH comprising EDTA and/or sodium azide. In other cases, a cartridge can be stored wet in such a buffer at room temperature provided that it is stored in conditions such that the matrix remains immersed in the buffer, i.e. such that it remains wet. The present disclosure also relates to methods for preparing a reusable cartridge for storage in between uses. For example, in some methods, the cartridge, following a prior analyte detection elution, is washed at least once in a buffer, such as 1-10% acetic acid, e.g., 1%, 5%, or 10% acetic acid, 1% formic acid, or 1% trifluoroacetic acid (TFA), and the matrix is then contacted with cryoprotectant, such as in a buffer of pH 7 or less, or in a buffer such as at pH 4-7, or in an acidic buffer such as at pH 4-6, pH 4-6.5, pH 4.5-6.5, pH 4.8-6.8, pH 5-7, pH 4-5, pH 5-6, pH 6-6.5, or pH 6-7. In some embodiments, the cryoprotectant comprises one or more of sucrose, trehalose, ethylene glycol, propylene glycol, glycerol, 2-methyl-2,4-pentanediol (MPD), or dimethyl sulfoxide (DMSO). In some embodiments, the cryoprotectant comprises sucrose and/or trehalose. In some embodiments, the cryoprotectant comprises trehalose. In some embodiments, the cryoprotectant-comprising buffer also comprises EDTA and/or sodium azide. In some embodiments, the cartridge is then allowed to dry. In some cases, the cartridge matrix is dried by heating to at least 30° C., such as to 30-45° C., 30-37° C., 30° C., or 37° C., for example, for at least 30 minutes, for 30-60 minutes, or for 60-120 minutes, then allowed to cool at room temperature for at least 1 hour, such as at least 2 hours, at least 4 hours, at least 8 hours, at least 12 hours, or overnight, before optionally storing at reduced temperature before re-use.

In some embodiments, a reusable cartridge is prepared by obtaining a cartridge comprising a matrix, or optionally adding a matrix to a cartridge, contacting the matrix with the detection molecules, allowing the molecules to become attached to the matrix, and optionally crosslinking the detection molecules to the matrix. In some cases, the excess, non-attached detection molecules are then removed. Before use, the cartridge may be equilibrated using appropriate buffers.

In some embodiments, the reusable cartridge is prepared with antibodies as detection molecules. In some such cases, the matrix may comprise a molecule that specifically recognizes portions of antibodies such as Fc domains or other constant regions, ideally allowing antibodies to bind to the matrix in regions that do not interfere with their antigen binding functions. For example, in some embodiments, purified or isolated antibodies are exposed to a matrix comprising, for instance, protein A, protein G, or protein L, or combinations of these such as protein A and protein G, allowed to bind to the matrix, with excess antibody then removed. In some cases, a crosslinker is used to cross-link the antibody detection molecules to the protein A, G, and/or L comprising matrix. Similarly, when other detection molecules are used, such as haptens or other binding agents of an analyte to be detected, they may also be crosslinked to the matrix.

In some embodiments, one type of detection molecule is attached to the matrix, such as, an antibody specific for a particular antigen. In other embodiments, two or more types of detection molecules may be attached to the matrix. In some cases, detection molecules recognizing more than one analyte can be included on a single matrix, such as two different antibodies targeting two different antigens, or several different antibodies targeting a group of analytes. In other cases, two or more different antibodies recognizing different parts of the same antigen may be attached to the matrix, for example. Thus, in some embodiments, the matrix can be bound by 2, 3, 4, 5, 10, 20, 100, or 2-5, 5-10, 10-20, 10-50, 10-100, or 50-100 different detection molecules, for example, so that the matrix is capable of recognizing a series or panel of analytes in a solution.

In some cases, the detection molecules are crosslinked to the matrix, for example, in order to prevent them from being leached from the matrix over repeated use. In some embodiments, the crosslinker is selected from dimethylpimelimidate (DMP), a cyanate ester, an NHS ester, azlactone, carbonyl diimidazole (CDI), maleimide, iodoacetyl, pyridyl disulfide, hydrazide, or a carbodiimide. In some embodiments, crosslinking is performed using DMP. In some embodiments, a crosslinking reaction with DMP is performed in the cartridge at a DMP concentration that is one-quarter to one-fifth of the usually recommended concentration for such a crosslinking reaction. Thus, for instance, DMP may be recommended for use at about 20-25 mM, but in the present case, less than 10 mM DMP may be used for crosslinking a protein detection molecule, such as an antibody, to a matrix. In some cases, 3-10 mM DMP, 3-7 mM DMP, 5-10 mM DMP, or 4-6 mM DMP may be used. In some cases, 5 mM DMP may be used. In some embodiments, lowering concentrations of crosslinking agents compared to the generally recommended concentrations improved the ability of the cartridges to be re-used without significantly impairing the ability of the cartridge matrix to detect analytes.

### Exemplary Uses of Reusable Cartridges for Detection of Polypeptides

The present disclosure also encompasses methods of using and re-using cartridges herein. In some embodiments, the analyte is a peptide or protein, for example. For instance, the analyte may be a peptide or protein that is specifically recognized by an antibody detection molecule. For instance, as described herein, one example protein analyte is an MHC-I complex molecule.

In some cases, an analyte such as a protein or other biological molecule may be detected in a biological solution, such as a bodily fluid (e.g. blood, plasma, urine, etc.) or a cell lysate. For example, methods herein may allow for enrichment of MHC-I complex molecules from biological fluids or cell lysates so that they can be further analyzed in subsequent steps such as chromatography and mass spectrometry.

In some embodiments, the solution is filtered prior to exposing it to the cartridge. For example, filtration may help to remove large particulates that could clog the matrix or interfere with its subsequent re-use. In some embodiments, the solution is filtered on a 0.1 to 1 μm filter, such as on a 0.1 μm, 0.2 μm, 0.22 μm, 0.3 μm, 0.4 μm, 0.45 μm, 0.5 μm, 0.6 μm, 0.7 μm, or 0.8 μm filter, or on a 0.1 to 0.5 μm filter, such as on a 0.1 to 0.5 μm filter, e.g. a 0.1 μm, 0.2 μm, 0.22 μm, 0.3 μm, 0.4 μm, 0.45 μm, or 0.5 μm filter. In some embodiments, where a biological fluid or cell lysate is used for analyte detection, the fluid is chilled, for example, on ice, or to a temperature of, for example 2-15° C. before it is contacted with the cartridge matrix. For example, in some embodiments, the sample is chilled to a temperature of 2-10° C. or 4-10° C. or 10-15° C. In some embodiments, a cryoprotectant is added to the solution, such as sucrose, glycerol, or trehalose, prior to lowering the temperature. In some embodiments, the solution is flash frozen after cryoprotectant is added. In some embodiments, a biological fluid or cell lysate is diluted prior to contacting with the cartridge matrix, for example, in an isotonic buffer. In some embodiments, the total protein concentration in a solution may be determined prior to contact with the matrix, such as in a bicinchoninic acid (BCA) assay.

In some embodiments, once an analyte has been eluted from a cartridge herein, it may be further analyzed by processes such as electrophoresis, chromatography, or structural analysis, or mass spectrometry or chromatography followed by mass spectrometry.

In some embodiments, the amount of analyte bound to the matrix may also be quantitated. For example, the amount of analyte may be determined by, for example, electrophoresis and densitometry, or by integration of a signal from analyte in a chromatography peak.

In some cases, the methods of use include detecting or enriching an analyte with a cartridge that has been previously used for detection or enrichment of the same or a similar analyte previously. In some cases, the cartridge has been used at least once before. In some cases, the cartridge has been used at least twice before. In some cases, the cartridge has been used at least three times before. In some cases, the cartridge has been used at least four times before. In some cases, the cartridge has been used at least five times before. In some cases, the cartridge has been used at least six times before. In some cases, the cartridge has been used at least seven times before. In some cases, the cartridge has been used at least eight times before. In some cases, the cartridge has been used at least nine times before.

In some cases, the use involves, after eluting the analyte, preparing the cartridge for a subsequent use, such as by washing it at least once in a buffer, such as 1-10% acetic acid, e.g., 1%, 5%, or 10% acetic acid, 1% formic acid, or 1% trifluoroacetic acid (TFA), and optionally contacting the matrix with cryoprotectant. The cryoprotectant may be in a buffer, such as in a buffer of pH 7 or less, or in a buffer such as at pH 4-7, or in an acidic buffer such as at pH 4-6, pH 4-6.5, pH 4.5-6.5, pH 4.8-6.8, pH 5-7, pH 4-5, pH 5-6, pH 6-6.5, or pH 6-7. In some embodiments, the cryoprotectant comprises one or more of sucrose, trehalose, ethylene glycol, propylene glycol, glycerol, 2-methyl-2,4-pentanediol (MPD), or dimethyl sulfoxide (DMSO). In some embodiments, the cryoprotectant comprises sucrose and/or trehalose. In some embodiments, the cryoprotectant comprises trehalose. In some embodiments, the cryoprotectant-comprising buffer also comprises EDTA and/or sodium azide. In some embodiments, the cartridge is then allowed to dry. In some cases, the cartridge matrix is dried by heating to at least 30° C., such as to 30-45° C., 30-37° C., 30° C., or 37° C., for example, for at least 30 minutes, for 30-60 minutes, or for 60-120 minutes, then allowed to cool at room temperature for at least 1 hour, such as at least 2 hours, at least 4 hours, at least 8 hours, at least 12 hours, or overnight, before optionally storing at reduced temperature before re-use.

### Kits Comprising Cartridges

The present disclosure also encompasses kits that comprise cartridges herein. In some embodiments, the cartridges have not yet been used. In other embodiments, they have been previously used at least 1, 2, 3, 4, 5, 6, 7, 8, 9, 10, 20, 50, or 100 times. In some embodiments, the kits comprise cartridges stored in a dry state as described above. In some embodiments, the kits comprise cartridges stored in a wet state as described above. In some embodiments, the kits further include instructions for use. In some embodiments, the kits further comprise reagents for detecting or enriching an analyte in a sample, such as equilibration, wash, and/or elution buffers. In some embodiments, the kits further comprise controls, such as blank cartridges without detection molecules or cartridges with a structurally similar detection molecule that is not intended to recognize the analyte. In some embodiments, the kits may be packaged for shipment to other locations. In some embodiments, the kits comprising the cartridges may be stored in a dry state, as described above, for at least 24 hours, 48 hours, at least 1 week, at least 2 weeks, at least 1 month, at least 3 months, or at least 6 months, in some cases at room temperature and in other cases at 2-8° C. In some embodiments, the kits comprising the cartridges may be stored in a wet state, as described above, for at least 24 hours, 48 hours, at least 1 week, at least 2 weeks, at least 1 month, at least 3 months, at least 6 months, or at least 1 year, in some cases at room temperature and in other cases at 2-8° C. In some embodiments, a kit may also comprise reagents used to clean and store a cartridge, such as buffers comprising one or more of a cryoprotectant, EDTA, and sodium azide. In some embodiments, a kit comprises a pack of cartridges, for example, of 10, 50, 100 (e.g. a 96-well plate comprising 96 well-cartridges; or a set of about 100 tube-shaped cartridges that fit into a 96-well plate system), 500, or 1000 cartridges.

The following examples provide further description of exemplary cartridges herein and methods of preparing, storing and using them, but are not intended to limit the scope of the disclosure herein.

### EXAMPLES

### Example 1. Preparing and Testing Reusable Cartridges

Cartridge Preparation

The following materials were obtained for preparing antibody-coated cartridges: 6 large AssayMAP™ protein A cartridges (Agilent), a Seahorse plate, Eppendorf PCR plates (deep well plates), PBS, TBS, and 25 mM Tris buffers, a 1% acetic acid elution buffer, and a solution of an anti-human HLA A, B, C antibody w6/32 (1 mg/mL in PBS). Cartridges were assembled into pre-defined rows in the plates and equilibrated with PBS, contacted with the antibody solution, to allow antibodies to bind to protein A on the cartridge matrix, washed, and then exposed to cross-linking reagents. Specifically, with the cartridges arranged in a multi-well plate, and using an automated liquid dispensing device (Agilent AssayMAP™ Bravo), the cartridges were first primed and equilibrated with PBS, using 250 μL PBS at a flow rate of 300 μL/min, then 150 PBS at a flow rate of 20 μL/min. Antibody solution was then added at 1 mL at a flow rate of 20 μL/min, then cartridges were washed with PBS to remove excess antibody.

Cartridges were then treated with cross-linking reagent to crosslink antibody to the protein A matrix. Cartridges were first primed and equilibrated with 200 mM triethanolamine (TEA) buffer pH 8.2, then treated with 5 mM DMP in a volume of 200 μL at a flow rate of 5 μL/min. Cartridges were then washed first with 250 μL TBS and second with 250 μL 25 mM Tris, each at a flow rate of 20 μL/min, then contacted with an elution buffer of 1% acetic acid (50 μL at 10 μL/min). Cartridges were then treated a second time with the acetic acid, Tris, and then TBS buffers (100 μL, 200 μL, and 200 μL volumes each at a flow rate of 20 μL/min), and placed in storage buffer of 190 μL TBS and 1 mM EDTA and 0.025% sodium azide, making sure that liquid remained above the top of the matrix in each cartridge column.

Certain cartridges were dried as follows, while others were stored in the storage buffer. A drying buffer of 20 mM His-acetate, 200 mM trehalose, pH 6 was prepared. 270 μL of the drying buffer was added to the cartridges selected for dry storage. The cartridges were then placed at 37° C. for 1 hour to dry, and then stored overnight at room temperature before use.

Cartridge Testing

Antibody-coated and crosslinked cartridges stored in a wet state and stored in a dry state before use, prepared as described above, were then tested for binding to analyte in solution in an AssayMap™ Bravo system. Specifically, 3 wet-stored and 3 dry-stored w6/32 antibody cartridges were used for testing. The cartridges were primed and equilibrated in TBS (150 μL at 300 μL/min for priming and then 100 μL at 20 μL/min for equilibration), the analyte solution was added (50 μL at 10 μL/min). HLA-containing solution was added to each of the wet or dry-stored w6/32 cartridges. The cartridges were then washed with 250 μL TBS at a flow rate of 20 μL/min, and then with 250 μL 25 mM Tris pH 8.0 at 20 μL/min, and subsequently eluted with 50 μL 1% acetic acid at 10 μL/min flow rate. Elutions were then loaded onto an acrylamide electrophoresis gel, which was then stained with SimplyBlue™ (ThermoFisher Scientific) by heating in a microwave for 1.5 minutes followed by a 10 minute shake and detstaining in water for 1 hour.

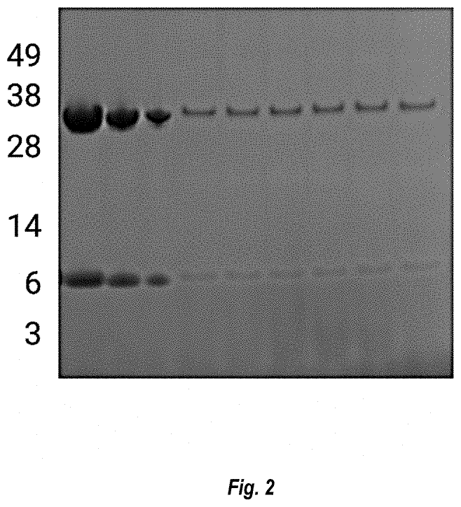

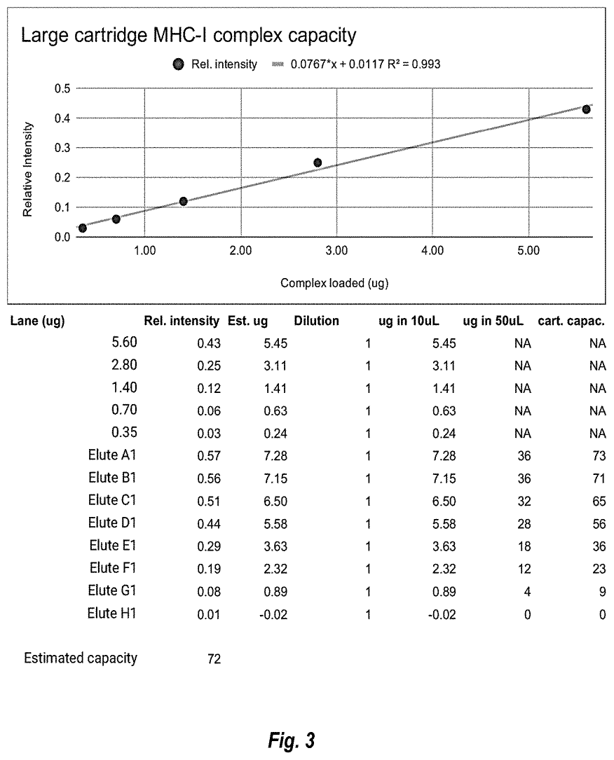

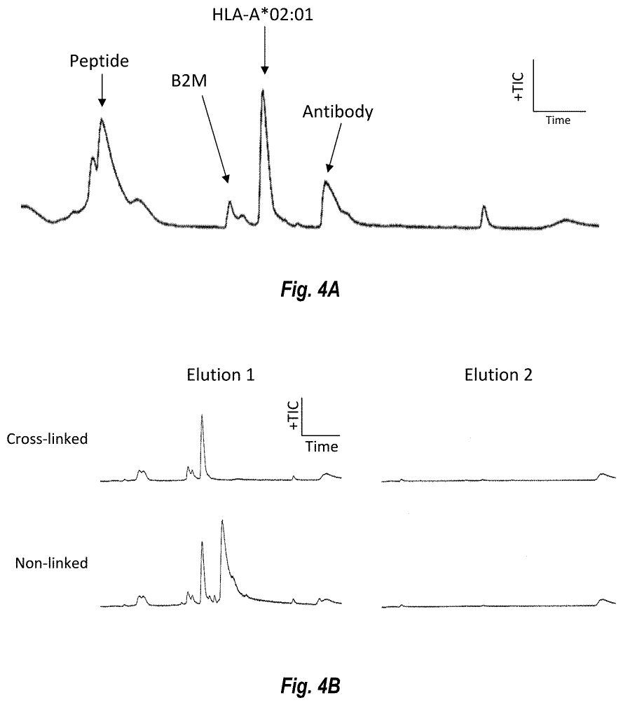

Gel lanes are as follows below and Results are as shown in FIG. 2.

| | | Volume |
|---|---|---|
| Lane | Sample | (μL) |
| 1 | HLA standard | 10 |
| 2 | HLA standard | 5 |
| 3 | HLA standard | 2 |
| 4 | Elution from w6/32 wet-stored cartridge 1 | 15 |
| 5 | Elution from w6/32 wet-stored cartridge 2 | 15 |
| 6 | Elution from w6/32 wet-stored cartridge 3 | 15 |
| 7 | Elution from w6/32 dry-stored cartridge 1 | 15 |
| 8 | Elution from w6/32 dry-stored cartridge 2 | 15 |
| 9 | Elution from w6/32 dry-stored cartridge 3 | 15 |

As can be seen in FIG. 2, HLA elution from wet and dry-stored cartridges (lanes 4-9 at the right of FIG. 2) appears equivalent, showing that the cartridges may be used after either wet or dry storage.

### Example 2: Use of Cartridges for Enrichment of MHC-I Complexes in Biological Samples

Materials and Peptide Synthesis

All chemicals were purchased from Sigma-Aldrich unless otherwise specified. Antibodies were purchased from CST (Danvers, Mass.) or Abcam (Burlingame, Calif.). The dTag13 degrader compound was purchased from Tocris. General plasticware was purchased from Corning, and AssayMAP™ plasticware from Agilent as specified for use with the AssayMAP™ Bravo in the user manual. Peptides were purchased from JPT Peptide Technologies (Berlin, Germany), dissolved in 50% ethylene glycol (Sigma), and stored at −20° C.

HLA and B2M Purification

Recombinant HLA alleles and β2M were over expressed in E. Coli, purified from inclusion bodies, and stored under denaturing conditions (6M Guanidine HCL, 25 mM Tris, pH 8.0) in −80° C. Briefly, β2M and HLA biomass pellets were resuspended in lysis buffer (PBS+1% Triton X-114) at a concentration of 5 mL/g. The resuspended pellets were subjected to microfluidization at 1000 bar twice. The resulting suspension was spun at 30,000 g for 20 min in an ultracentrifuge. The pellets were collected and washed with 500 mL of lysis buffer and centrifuged at 30,000 g for 20 min. The pellet was collected and washed a second time as described above. The purified inclusion bodies were then dissolved in denaturing buffer (20 mM MES, pH 6.0, 6M Guanidine HCl) at a concentration of 10 mL/g. The suspension was then stirred at 4° C. overnight. The dissolved pellet was centrifuged at 40,000 g for 60 min and the supernatant was collected and filtered through a 0.22 μm filter. The concentration was determined using a BCA assay. Samples were snap frozen and stored at −80° C. prior to use in generating the MHC-I complexes.

Generation of Recombinant MHC-I Complexes

In a 5 L reaction, the selected peptide (0.01 mM), oxidized and reduced glutathione (0.5 mM and 4.0 mM, respectively), recombinant HLA alleles (0.03 mg/ml) and β2M (0.01 mg/ml) were all combined in refold buffer (100 mM Tris, pH 8.0, 400 mM L-Arginine, 2 mM EDTA). The refold mixtures were stirred for 4 days at 4° C. The refold solution was filtered through a 0.22 μm filter, concentrated and buffer exchanged by tangential flow filtration (TFF) (Millipore) into 25 mM Tris, pH 7.5. The protein components were then analyzed by LC/MS to ensure that the HLA was in the appropriate reduced state. The refolded MHC-I complex was purified by ion exchange chromatography using a 5 mL HiTrap™ Q HP column on an AKTA Pur FPLC. The column was equilibrated with 10 column volumes (CV) of 25 mM Tris, pH 7.5 at 5 mL/min flow rate. The MHC-I complex was loaded on the column at a 5 mL/min flow rate and eluted using 0-60% 25 mM Tris, pH 7.5, 1 M NaCl gradient over 30 CV. Fractions across the eluted peak were run on SDS-PAGE and those fractions containing a β2M and HLA band were pooled. Pooled fractions were exchanged into storage buffer (25 mM Tris, pH 8.0, 150 mM NaCl). Protein concentration was determined by UV absorbance at 280 nm and samples were snap frozen and stored at −80° C.

Adherent and Suspension Cell Culture

MC38 cells were obtained as frozen stocks and immediately cultured into working stocks and frozen in growth media +10% DMSO. MC38's were grown in RPMI-1640, 10% FBS, 2 mM glutamine, 1X pen-strep, and 25 mM HEPES. Cells were passaged with an 18 hr doubling time, at 37° C. and 5% CO₂. GRANTA-519 (GRANTA) cells were also cultured into working stocks as above. GRANTA cells were cultured in RPMI-1640, 10% FBS, 2 mM glutamine, and 1X pen-strep. Cells were passaged with a 48 hr doubling time, at 37° C. and 5% CO₂ in an Infors Minitron at 110 rpm.

Cell Treatment and Harvest

Cells were treated and incubated at 37° C. and 5% CO₂ (adherent MC38 cells), and 110 rpm (suspension GRANTA cells). To harvest adherent MC38 cells, media was aspirated and cold Accutase (Innovative Cell Technologies, Inc.) immediately added to plates. Plates were allowed to sit for 5 mins at room temperature prior to using agitation to lift cells off the plate. The Accutase cell mixture was then added to falcon tubes containing growth media, cells were counted using a ViCell XR and the viable cell concentration per mL determined, and 250 million cells were then transferred to 50 mL falcon tubes per condition. To harvest suspension GRANTA cells, cells were counted and transferred as above. All cells were then pelleted via centrifugation, the supernatant removed, and the pellet flash frozen in liquid nitrogen before being placed at −80° C.

Cell Lysis and Storage

Cell pellets were removed from −80° C. and quickly thawed in a 37° C. water bath before being placed on ice. 250 million cell GRANTA pellets were lysed in 5 mL non-denaturing OG detergent buffer (PBS, 0.25% sodium deoxycholate, 0.2 mM iodoacetamide, 1 mM EDTA, 1% octyl-beta-d glucopyranoside (OG), and 1X protease+phosphatase inhibitors (Sigma)) and the lysate was transferred to one 5 mL eppendorf tube. 250 million cell MC38 pellets were lysed in 10 mL OG buffer each and transferred to two 5 mL eppendorf tubes. Lysates were placed on ice for 30 mins then spun down 20,000 g for 60 min at 4° C. to clarify. Clarified lysates were immediately decanted into 50 mL falcon tube vacuum filters (0.45 μm, Corning) and filtered under gentle vacuum. Filtered solutions were transferred to respective 15 mL falcon tubes on ice in 4 mL aliquots (an entire GRANTA 250 M cell lysis or half of a MC38 250 M cell lysis), followed by 1.33 mL each of 50% glycerol and 1 M sucrose in (for a final concentration of 10% glycerol and 200 mM sucrose). Tubes were mixed by inversion until homogenous, 10 μL was removed for BCA analysis, and 30 μL for western blotting and in-gel digest (for global proteomics). Falcon tubes were then flash frozen, and placed at −80° C.

Cartridge Preparation, Storage, and Re-Use

Cartridge cross-linking was carried out as follows. Briefly, dry protein A cartridges were primed in PBS before loading 1 mg of antibody at 1 mg/mL, followed by washing with PBS (for all cartridge sizes, priming occurred at 300 μL/min. Washing and loading small cartridges occurred at 10 μL and 5 μL/min respectively. Washing and loading large cartridges occurred at 20 μL/min). Cartridges were then equilibrated into 200 mM triethanolamine (TEA, Sigma), loaded with 5 mM Dimethyl Pimelimidate (DMP, Sigma) in TEA, pH 8.2, over 40 minutes at room temperature, then washed in succession with TBS, 25 mM Tris, pH 8.0 (Tris buffer), 1% acetic acid, Tris buffer, and finally TBS. Cartridges were then stored in empty cartridge racks filled with TBS, 1 mM EDTA, 0.025% sodium azide at 4° C., then parafilmed. To make dried cartridges, the cartridge buffer was exchanged into 20 mM Histidine, 200 mM trehalose, pH 6.0, placed at 37° C. for 1 hr, then left at room temperature in the dark for at least 18 hrs to complete drying. Dried cartridges were then reconstituted in the same manner as dry protein A cartridges.

Re-used cartridges were transferred to the AssayMAP™ Bravo, the neck of each cartridge was dried with a cotton swab (if transferred from 4° C.), then primed with water, primed with 1% acetic acid, and washed with water before reuse.

MEW Enrichment Using Cartridges

Diluted, frozen cell lysates were removed from the −80° C. freezer on dry ice and quickly thawed in a 37° C. water bath, then placed on wet ice without mixing. Cross-linked cartridges were removed from the refrigerator and transferred to the AssayMAP™ system, the cup dabbed dry using a Qtip, and primed with water, primed with 1% acetic acid, and washed with water.

500 μL from each lysate was transferred to 0.45 μm Costar spin filter tubes, spun down at 16,000 g for 1 min at 4° C., then placed on ice. Spin filter tubes were checked for retention of lysate on top of the filter. If the retained lysate volume was greater than 10-20 material was not used for the cartridge experiments as it was considered to likely clog the cartridges. Return flow-throughs to respective 15 mL falcon tubes were stored on ice. The sample was cooled to 10° C.

From each 8.3 mL falcon tube, 1.1 mL per well was transferred to four wells of a Deepwell™ sample plate (3.9 mL sample remained in the falcon tube). The AssayMAP™ Bravo system was used to prime and equilibrate cartridges and load samples as follows: cartridge TBS priming (150 μL at 250 μL/min), TBS equilibration (100 μL at 20 μL/min), and sample loading (1 mL at 20 μL/min). The flow-through plate was replaced and an additional 800 μL sample was added to each of the four wells. Further sample loading (800 μL at 20 μL/min), followed by TBS wash (250 μL at 25 μL/min), Tris wash (250 μL at 25 μL/min), and 1% acetate elution (60 μL at 10 μL/min) was conducted. 10 μL from each well was removed to validate MHC-I complex enrichment by Coomassie-stained electrophoresis gel. The remaining 4×50 μL was combined into a single, 1.5 mL volume tube, flash frozen, and placed at −80° C. Cartridges were then primed with 1% acetic acid, and washed with Tris buffer and TBS before storage in empty cartridge racks filled with TBS, 1 mM EDTA, 0.025% sodium azide at 4° C., then parafilmed.

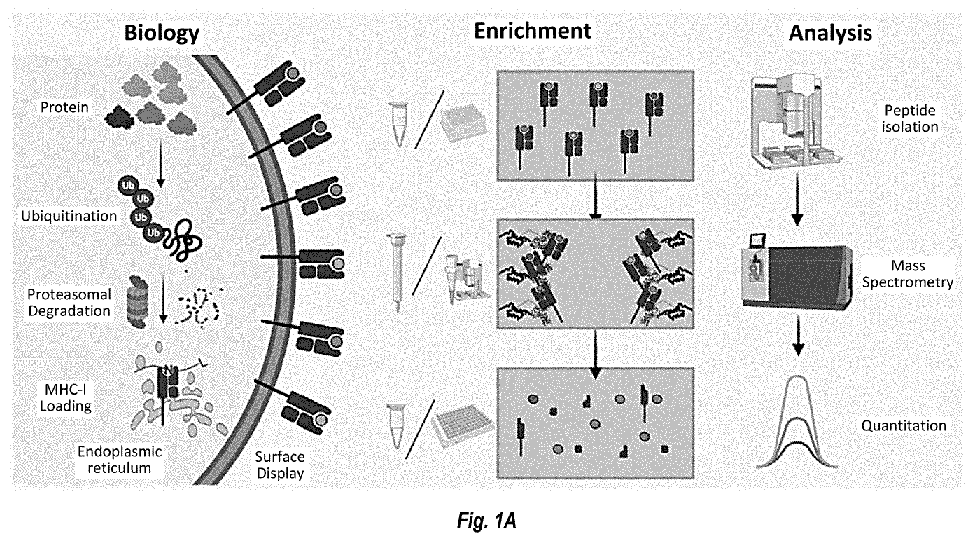

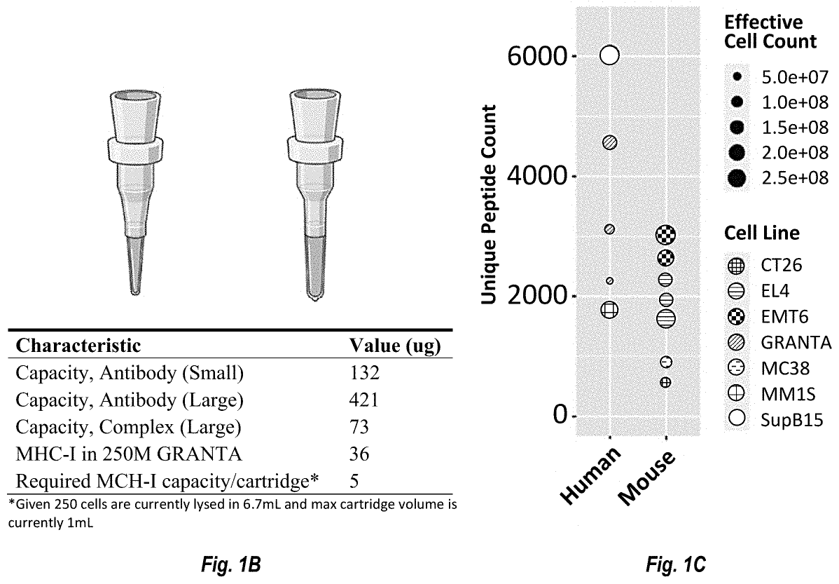

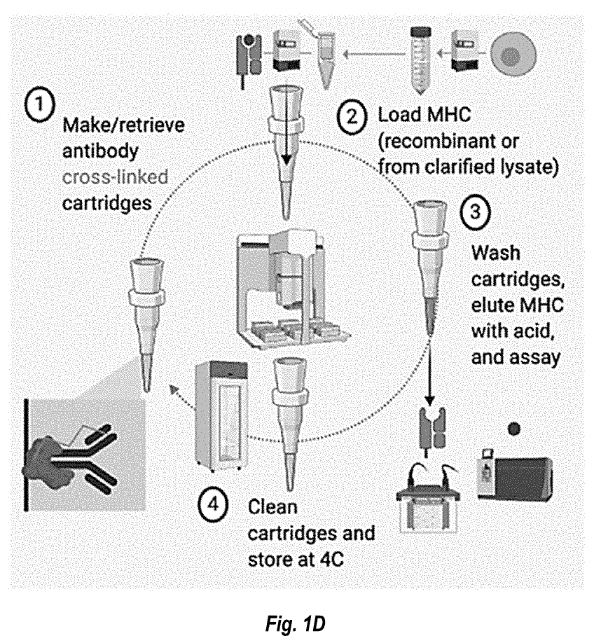

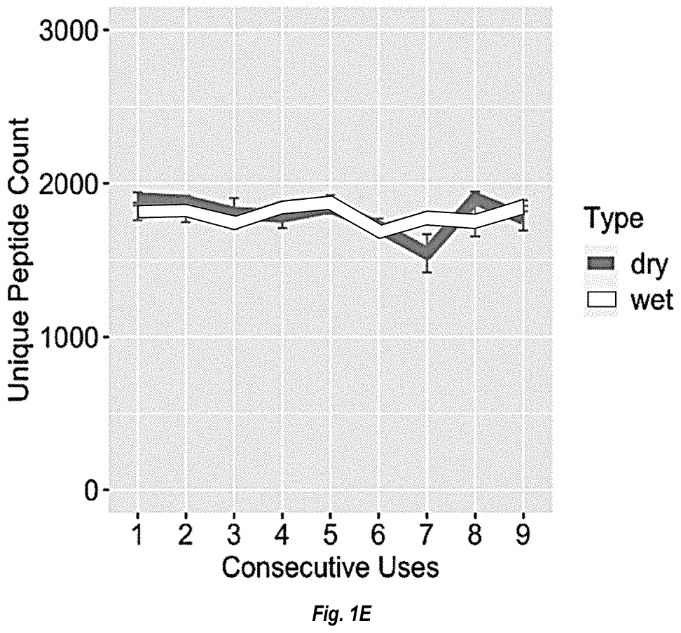

FIG. 1D shows a workflow for preparation and use of the cartridges for detecting MHC-I complexes in a cell lysate. Initially we found that clogging of the cartridges after the first use prevented our ability to re-use them. However by reducing the amount of the DMP crosslinker (e.g. from 25 mM to 5 mM), using a larger lysate filter, diluting the filtered lysate 1.33-fold with glycerol and sucrose, and lowering the temperature of the lysate during enrichment, we found that precipitation was minimized and the cartridges were no longer obstructed on repeat use.

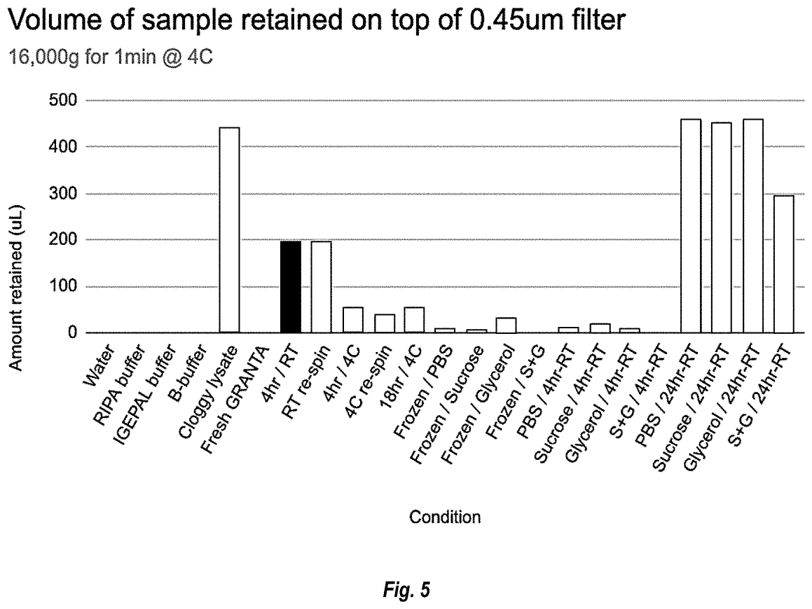

Specifically, as shown in FIG. 5, under the conditions described herein (500 μL placed on a 0.45 μm Costar filter and spun at 16,000 g for 1 min at 4° C.), water and lysis buffers show no volume retained (negative control), while room temperature day-old MC38 lysate (50 M cells per mL of B-buffer, positive control), shows retention of over 400 μL. Fresh GRANTA lysate (50 M cells per mL of B-buffer) shows no retention, while lysate stored for 4 hours at room temperature (typical, un-optimized loading conditions), shows a major increase in viscosity. Lowering the temperature to 4° C. reduces aggregation in a manner that is maintained over 18 hrs. If lysate is flash frozen with PBS, sucrose (1M starting, 200 mM final), or glycerol (50% glycerol starting, 10% final) and then thawed, viscosity is low, even after 4 hr at room temperature (however storage for 24 hr at room temperature leads to a large increase in viscosity). Combining sucrose and glycerol preservation (leading to an even more dilute lysate solution) allows for a freeze-thaw cycle with no apparent retention, both immediately, and after 4 hrs at room temperature.

Using this optimized workflow we demonstrated that a single cartridge could be used to enrich lysates in the above assay at least nine times with little decrease in unique peptides observed or change in the composition of peptides detected (see FIG. 1E, FIGS. 6F-6H). This finding held true even if the antibody cross linked cartridges had been dried after cross-linking, stored in a dry state, and then re-wetted before use.

Unless stated otherwise herein, all citations and references are incorporated by reference herein in their entirety.

### REFERENCES

1. Leko, V. & Rosenberg, S. A. Identifying and Targeting Human Tumor Antigens for T Cell-Based Immunotherapy of Solid Tumors. Cancer Cell (2020) doi:10.1016/j.cce11.2020.07.013.
2. Laumont, C. M. et al. Global proteogenomic analysis of human MHC class I-associated peptides derived from non-canonical reading frames. Nat. Commun. 7, 10238 (2016).
3. Laumont, C. M. & Perreault, C. Exploiting non-canonical translation to identify new targets for T cell-based cancer immunotherapy. Cell. Mol. Life Sci. 75, 607-621 (2018).
4. Kong, Y. et al. Transposable element expression in tumors is associated with immune infiltration and increased antigenicity. Nat. Commun. 10, 5228 (2019).
5. Ouspenskaia, T. et al. Thousands of novel unannotated proteins expand the MHC I immunopeptidome in cancer. bioRxiv 2020.02.12.945840 (2020) doi:10.1101/2020.02.12.945840.
6. Ott, P. A. et al. An immunogenic personal neoantigen vaccine for patients with melanoma. Nature 547, 217-221 (2017).
7. Yadav, M. et al. Predicting immunogenic tumour mutations by combining mass spectrometry and exome sequencing. Nature 515, 572-576 (2014).
8. Sahin, U. et al. Personalized RNA mutanome vaccines mobilize poly-specific therapeutic immunity against cancer. Nature 547, 222 (2017).
9. Fernandez-Poma, S. M. et al. Expansion of Tumor-Infiltrating CD8+ T cells Expressing PD-1 Improves the Efficacy of Adoptive T-cell Therapy. Cancer Res. 77, 3672-3684 (2017).
10. Locke, F. L. et al. Phase 1 Results of ZUMA-1: A Multicenter Study of KTE-C19 Anti-CD19 CAR T Cell Therapy in Refractory Aggressive Lymphoma. Mol. Ther. J. Am. Soc. Gene Ther. 25, 285-295 (2017).
11. Lundegaard, C. et al. NetMHC-3.0: accurate web accessible predictions of human, mouse and monkey MHC class I affinities for peptides of length 8-11. Nucleic Acids Res. 36, W509-512 (2008).
12. O'Donnell, T. J. et al. MHCflurry: Open-Source Class I MEW Binding Affinity Prediction. Cell Syst. 7, 129-132.e4 (2018).
13. Peters, B., Nielsen, M. & Sette, A. T Cell Epitope Predictions. Annu. Rev. Immunol. 38, 123-145 (2020).
14. Lanoix, J. et al. Comparison of the MHC I Immunopeptidome Repertoire of B-Cell Lymphoblasts Using Two Isolation Methods. PROTEOMICS 18, 1700251 (2018).
15. Purcell, A. W., Ramarathinam, S. H. & Ternette, N. Mass spectrometry-based identification of MHC-bound peptides for immunopeptidomics. Nat. Protoc. 14, 1687-1707 (2019).
16. Capietto, A.-H. et al. Mutation position is an important determinant for predicting cancer neoantigens. J. Exp. Med. 217, (2020).
17. Chong, C. et al. High-throughput and Sensitive Immunopeptidomics Platform Reveals Profound InterferonG-Mediated Remodeling of the Human Leukocyte Antigen (HLA) Ligandome. Mol. Cell. Proteomics MCP 17, 533-548 (2018).
18. Stopfer, L. E., Mesfin, J. M., Joughin, B. A., Lauffenburger, D. A. & White, F. M. Multiplexed relative and absolute quantitative immunopeptidomics reveals MHC I repertoire alterations induced by CDK4/6 inhibition. Nat. Commun. 11, 2760 (2020).
19. Pfammatter, S. et al. Extending the Comprehensiveness of Immunopeptidome Analyses Using Isobaric Peptide Labeling. Anal. Chem. (2020) doi:10.1021/acs.analchem.0c01545.
20. Rose, C. M. et al. TomahaqCompanion: A Tool for the Creation and Analysis of Isobaric Label Based Multiplexed Targeted Assays. J. Proteome Res. 18, 594-605 (2019).
21. Erickson, B. K. et al. A Strategy to Combine Sample Multiplexing with Targeted Proteomics Assays for High-Throughput Protein Signature Characterization. Mol. Cell 65, 361-370 (2017).
22. Gallien, S., Kim, S. Y. & Domon, B. Large-Scale Targeted Proteomics Using Internal Standard Triggered-Parallel Reaction Monitoring (IS-PRM). Mol. Cell. Proteomics MCP 14, 1630-1644 (2015).
23. Fulton, S. et al. A high-throughput microchromatography platform for quantitative analytical scale protein sample preparation. J. Lab. Autom. 16, 457-467 (2011).
24. Bassani-Sternberg, M., Pletscher-Frankild, S., Jensen, L. J. & Mann, M. Mass Spectrometry of Human Leukocyte Antigen Class I Peptidomes Reveals Strong Effects of Protein Abundance and Turnover on Antigen Presentation. Mol. Cell. Proteomics 14, 658-673 (2015).
25. Nabet, B. et al. The dTAG system for immediate and target-specific protein degradation. Nat. Chem. Biol. 14, 431-441 (2018).
26. Moser, S. C., Voerman, J. S. A., Buckley, D. L., Winter, G. E. & Schliehe, C. Acute Pharmacologic Degradation of a Stable Antigen Enhances Its Direct Presentation on MHC Class I Molecules. Front. Immunol. 8, (2018).
27. Jensen, S. M., Potts, G. K., Ready, D. B. & Patterson, M. J. Specific MHC-I Peptides Are Induced Using PROTACs. Front. Immunol. 9, (2018).
28. Liao, Y., Smyth, G. K. & Shi, W. The R package Rsubread is easier, faster, cheaper and better for alignment and quantification of RNA sequencing reads. Nucleic Acids Res. 47, e47-e47 (2019).
29. Love, M. I., Huber, W. & Anders, S. Moderated estimation of fold change and dispersion for RNA-seq data with DESeq2. Genome Biol. 15, (2014).
30. Wickham, H. et al. Welcome to the Tidyverse. J. Open Source Softw. 4, 1686 (2019).
31. R: The R Project for Statistical Computing. https://www.r-project.org/.
32. Phu, L. et al. Improved Quantitative Mass Spectrometry Methods for Characterizing Complex Ubiquitin Signals. Mol. Cell. Proteomics MCP 10, (2011).
33. Hos, B. J. et al. Identification of a neo-epitope dominating endogenous CD8 T cell responses to MC-38 colorectal cancer. Oncolmmunology 9, 1673125 (2020).
34. Blachère, N. E., Darnell, R. B. & Albert, M. L. Apoptotic Cells Deliver Processed Antigen to Dendritic Cells for Cross-Presentation. PLoS Biol. 3, (2005).
35. Dutoit, V. et al. Multiepitope CD8+ T cell response to a NY-ESO-1 peptide vaccine results in imprecise tumor targeting. J. Clin. Invest. 110, 1813-1822 (2002).
36. Dao, T. et al. An immunogenic WT1-derived peptide that induces T cell response in the context of HLA-A*02:01 and HLA-A*24:02 molecules. Oncoimmunology 6, (2016).
37. Aurisicchio, L. et al. Poly-specific neoantigen-targeted cancer vaccines delay patient derived tumor growth. J. Exp. Clin. Cancer Res. CR 38, (2019).
38. Vogel, R. et al. Mass Spectrometry Reveals Changes in MHC I Antigen Presentation After Lentivector Expression of a Gene Regulation System. Mol. Ther. Nucleic Acids 2, e75 (2013).
39. Yu, Q. et al. Sample multiplexing for targeted pathway proteomics in aging mice. Proc. Natl. Acad. Sci. U.S.A 117, 9723-9732 (2020).
40. Martinez, T. F. et al. Accurate annotation of human protein-coding small open reading frames. Nat. Chem. Biol. 16, 458-468 (2020).
41. Smith, C. C. et al. Endogenous retroviral signatures predict immunotherapy response in clear cell renal cell carcinoma. J. Clin. Invest. 128, 4804-4820 (2019).
42. Wang, G. et al. Multiplexed activation of endogenous genes by CRISPRa elicits potent antitumor immunity. Nat. Immunol. 20, 1494-1505 (2019).
43. Krajcovicova, S., Jorda, R., Hendrychova, D., Krystof, V. & Soural, M. Solid-phase synthesis for thalidomide-based proteolysis-targeting chimeras (PROTAC). Chem. Commun. 55, 929-932 (2019).
44. Clift, D. et al. A Method for the Acute and Rapid Degradation of Endogenous Proteins. Cell 171, 1692-1706.e18 (2017).
45. Schlaeppi, J.-M. et al. A semi-automated large-scale process for the production of recombinant tagged proteins in the Baculovirus expression system. Protein Expr. Purif. 50, 185-195 (2006).
46. Thompson, A. et al. TMTpro: Design, Synthesis, and Initial Evaluation of a Proline-Based Isobaric 16-Plex Tandem Mass Tag Reagent Set. Anal. Chem. 91, 15941-15950 (2019).

What is claimed is:

1. A reusable cartridge for detecting an analyte in a solution, wherein the cartridge comprises:
a) a matrix,
b) detection molecules attached to the matrix, wherein the detection molecules specifically bind to the analyte, and wherein the detection molecules are optionally cross-linked to the matrix; and
c) at least one cryoprotectant,
wherein the cartridge can be used at least 10, 20, 50, or 100 times for detection of the analyte in a solution, and wherein, after each use the matrix is optionally stored with cryoprotectant in a dry state.

2. The reusable cartridge of claim 1, wherein the cartridge is a tube open at both ends, a tube open at one end, a well, a plate with or without wells, or a chip capable of containing the matrix.

3. The reusable cartridge of claim 1 or 2, wherein the matrix comprises particles (e.g. beads, grains, chips, or pellets) comprising silica or agarose, wherein the particles are optionally magnetic.

4. The reusable cartridge of any one of claims 1-3, wherein the matrix comprises protein A, protein G, and/or protein L.

5. The reusable cartridge of any one of claims 1-4, wherein the detection molecules are antibodies.

6. The reusable cartridge of any one of claims 1-5, wherein the detection molecules are cross-linked to the matrix.

7. The reusable cartridge of claim 6, wherein the detection molecules are cross-linked to the matrix with a crosslinker selected from selected from dimethylpimelimidate (DMP), a cyanate ester, an NHS ester, azlactone, carbonyl diimidazole (CDI), maleimide, iodoacetyl, pyridyl disulfide, hydrazide, or a carbodiimide.

8. The reusable cartridge of any one of claims 1-7, wherein the analyte detectable by the detection molecules on the matrix is a protein or peptide.

9. The reusable cartridge of claim 8, wherein the analyte is a major histocompatibility complex I (MHC-I) molecule.

10. The reusable cartridge of any one of claims 1-9, wherein the cartridge can be used at least 10 times for detection of a protein analyte in a cell lysate or biological fluid.

11. The reusable cartridge of any one of claims 1-10, which is stored in the dry state.

12. The reusable cartridge of claim 11, wherein the cartridge can be used at least 10 times for detection of a protein analyte in a cell lysate or biological fluid after storage in the dry state after each use.

13. The reusable cartridge of claim 11, wherein the cartridge can be used at least 20 times for detection of a protein analyte in a cell lysate or biological fluid after storage in the dry state after each use.

14. The reusable cartridge of claim 11, wherein the cartridge can be used at least 50 times for detection of a protein analyte in a cell lysate or biological fluid after storage in the dry state after each use.

15. The reusable cartridge of claim 11, wherein the cartridge can be used at least 100 times for detection of a protein analyte in a cell lysate or biological fluid after storage in the dry state after each use.

16. The reusable cartridge of any one of claims 11-15, wherein the matrix is stored at acidic pH.

17. The reusable cartridge of any one of claims 11-16, wherein the matrix is stored at 2-8° C.

18. The reusable cartridge of any one of claims 1-10, wherein the matrix is stored in a wet state.

19. The reusable cartridge of claim 18, wherein the matrix is stored at acidic pH.

20. The reusable cartridge of claim 18 or 19, wherein the matrix is stored at 2-8° C.

21. The reusable cartridge of any one of claims 1-20, wherein the cryoprotectant comprises one or more of sucrose, trehalose, ethylene glycol, propylene glycol, glycerol, 2-methyl-2,4-pentanediol (MPD), or dimethyl sulfoxide (DMSO).

22. A method of preparing a previously used cartridge according to any one of claims 1-21 for re-use, comprising, after analyte has been eluted from the cartridge, washing the matrix in 1-10% acetic acid, 1% formic acid, or 1% trifluoroacetic acid (TFA), washing the matrix in a buffer comprising cryoprotectant, and allowing the matrix to dry.

23. The method of claim 22, wherein the cryoprotectant comprises one or more of sucrose, trehalose, ethylene glycol, propylene glycol, glycerol, 2-methyl-2,4-pentanediol (MPD), or dimethyl sulfoxide (DMSO).

24. The method of claim 22 or 23, wherein the matrix is allowed to dry by heating the cartridge to at least 30° C., followed by storage at room temperature for at least 1 hour.

25. The method of claim 24, wherein the matrix is allowed to dry by heating the cartridge to 37° C., followed by storage at room temperature for at least 1 hour.

26. The method of any one of claims 22-25, wherein, after the matrix is allowed to dry, the cartridge is stored in a dry state at 2-8° C. until re-use.

27. A method of preparing a reusable cartridge according to any one of claims 1-21, the method comprising obtaining a cartridge comprising a matrix, contacting the matrix with the detection molecules, and crosslinking the detection molecules to the matrix.

28. A method of detecting an analyte in a solution, comprising contacting the matrix of the reusable cartridge according to any one of claims 1-21 with a solution comprising the analyte, and optionally eluting the analyte from the cartridge.

29. The method of claim 28, wherein the analyte is a peptide or protein.

30. The method of claim 29, wherein the analyte is an MHC-I molecule.

31. The method of any one of claims 28-30, wherein the solution is a biological fluid or cell lysate.

32. The method of claim 31, wherein the solution is filtered or treated to remove cell debris and membranous materials prior to contact with the matrix.

33. The method of any one of claims 28-30, wherein the cartridge has previously been used to detect the analyte at least 1, 2, 3, 4, 5, 6, 7, 8, 9, 10, 20, 50, or 100 times prior to its use in the method.

34. The method of any one of claims 28-33, wherein, following elution of analyte, the cartridge is treated by the method of any one of claims 22-26.

35. A kit comprising at least one reusable cartridge according to any one of claims 1-21.

36. The kit of claim 35, further comprising: (a) at least one buffer; (b) at least one control cartridge without detection molecule or with a control detection molecule; (c) reagents for preparing the cartridge for storage and reuse; and/or (d) instructions for use.
